(19) United States
(12) Patent Application Publication
(10) Pub. No.: US 2018/0303729 A1
(43) Pub. Date: Oct. 25, 2018

(54) DENTIFRICE COMPOSITIONS HAVING IMPROVED AESTHETICS

(71) Applicant: The Procter & Gamble Company, Cincinnati, OH (US)

(72) Inventor: Zhisheng CHEN, Beijing (CN)

(21) Appl. No.: 15/957,223

(22) Filed: Apr. 19, 2018

(30) Foreign Application Priority Data
Apr. 21, 2017 (WO) CN2017/081323

(51) Int. Cl. A61K 8/19; A61Q 11/00; A61K 8/29; A61K 8/25; A61K 8/26; A61K 8/21

(52) U.S. Cl. CPC A61K 8/19; A61Q 11/00; A61K 8/29; A61K 8/25; A61K 2800/92; A61K 8/21; A61K 2800/436; A61K 2800/412; A61K 8/26

(57) ABSTRACT

Dentifrice composition comprising at least one phase containing activated carbon and interference pigment.

### FIELD OF THE INVENTION

The present invention relates to dentifrice compositions having improved aesthetics.

### BACKGROUND OF THE INVENTION

There is a continuing need to improve aesthetics in dentifrice. Aesthetics are important for various reasons including connoting certain functional benefits as well as a premium experience. Examples activated carbon containing toothpaste has been commercialized. Often these activated carbon containing toothpastes have a black color appearance and often provided as a dual phase formulation wherein the other phase is a stark white (e.g., containing titanium dioxide). This black and white contrast provides a stark color contrast and has an impactful impact to users. However, there is a continuing need to improve this aesthetic impression to users.

### SUMMARY OF THE INVENTION

Applicants have surprisingly discovered that the use of interference pigments in dentifrice, especially against the black backdrop provided by activated carbon containing formulations. Consumer testing demonstrates a significant preference for those dentifrice formulations containing such interference pigments as compared to at least one commercialized product.

One aspect of the invention provides a dentifrice composition comprising 0.001% to 2% of an interference pigment by weight of the composition.

Another aspect of the invention provides for a method of brushing teeth comprising the step of contacting teeth with aforementioned dentifrice composition.

These and other features of the present invention will become apparent to one skilled in the art upon review of the following detailed description when taken in conjunction with the appended claims.

### DETAILED DESCRIPTION OF THE INVENTION

### Definitions

As used herein, the term “average molecular weight” refers to the average molecular weight as determined using gel permeation chromatography according to the protocol found in Colloids and Surfaces A. Physico Chemical & Engineering Aspects, Vol. 162, 2000, pg. 107-121. Unless otherwise specified, all molecular weight values herein refer to the weight average molecular weight and expressed in g/mol.

The term “comprising” as used herein means that steps and ingredients other than those specifically mentioned can be added. This term encompasses the terms “consisting of” and “consisting essentially of.” The compositions of the present invention can comprise, consist of, and consist essentially of the essential elements and limitations of the invention described herein, as well as any of the additional or optional ingredients, components, steps, or limitations described herein.

The term “orally acceptable carrier” as used herein means a suitable vehicle or ingredient, which can be used to form and/or apply the present compositions to the oral cavity in a safe and effective manner.

The term “dentifrice” as used herein means paste, gel, powder, tablets, or liquid formulations, unless otherwise specified, that are used to clean the surfaces of the oral cavity.

The term “teeth” as used herein refers to natural teeth as well as artificial teeth or dental prosthesis.

The term “total water content” as used herein means both free water and water that is bound by other ingredients in the dentifrice composition.

The term “water-insoluble” as used herein with respect to flavorants refers to flavor oils which has a water solubility of less than about one grams per 100 grams of water at 25° C.

All percentages, parts and ratios are based upon the total weight of the compositions of the present invention, unless otherwise specified. All such weights as they pertain to listed ingredients are based on the active level and, therefore do not include solvents or by-products that may be included in commercially available materials, unless otherwise specified.

As used herein, the articles including “a” and “an” when used in a claim, are understood to mean one or more of what is claimed or described.

As used herein, the terms “comprise”, “comprises”, “comprising”, “include”, “includes”, “including”, “contain”, “contains”, and “containing” are meant to be non-limiting, i.e., other steps and other sections which do not affect the end of result can be added. The above terms encompass the terms “consisting of” and “consisting essentially of”.

As used herein, the words “preferred”, “preferably” and variants refer to embodiments of the invention that afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the invention.

### Activated Carbon Phase

The dentifrice composition of the present invention comprises at least one phase of an activated carbon comprising composition, i.e., an activated carbon composition portion. Preferably the dentifrice composition comprises at least one activated carbon composition portion and at least one non-activated carbon composition portion. An example of a non-activated carbon composition portion is one that has a white color imparted by the presence of titanium dioxide (and is preferably free or substantially free of activated carbon). The dentifrice composition can be a single phase, two phases, three phases, four phases, or more. Preferably the dentifrice composition has at least two phases wherein one phase is an activated carbon composition portion and the second phase is a non-activated carbon composition portion. The activated carbon composition portion may comprise from 0.001% to 2%, preferably from 0.01% to 1%, more preferably from 0.05% to 0.5%, yet more preferably from 0.07% to 0.2%, of an activated carbon by weight of the activated carbon composition portion. Examples weight percentage of activated carbon contained in the activated carbon composition portion include 0.07, 0.09, 0.1, 0.15, 0.2, or 0.3.

One example of activated carbon is from CPS (Huhzou) Biotechnology Co., Ltd., where bamboo as a raw material is heated 1000° C. to 1200° C., and subjected to high temperature for one month, for carbonization. Thereafter, it is cooled down naturally to ambient temperature. The resulting charcoal block is milled to the desired particle size, and passed through a mesh. Activation is through a fluidized bed. One example of activated carbon is CAS No. 8021-96-6 (“charcoal powder”). The particles of activated carbon may have diameters under 10 microns, having an absorption capacity of up to 1200 mg per gram. Preferably the activated carbon has an iodine absorption capacity greater than 500 mg/g, more preferably greater than 1000 mg/g. Preferably the activated carbon has a specific surface area greater than 500 m²/g, more preferably greater than 1000 m²/g. Activated carbon may help with stain absorbance and/or deep cleaning of the oral cavity, especially teeth.

The non-activated carbon composition portion may comprise from 0.01% to 3%, preferably from 0.05% to 2%, more preferably from 0.1% to 1%, of a titanium dioxide (TiO₂) contained in the non-activated carbon composition portion. Examples weight percentage of titanium dioxide contained in the non-activated carbon composition portion include 0.3, 0.4, 0.5, 0.6, 0.7, or 0.8.

The at least one phase of the dentifrice composition is an activated carbon composition portion, wherein the activated carbon composition portion comprises an interference pigment. Preferably the dentifrice composition comprises a non-activated carbon composition, more preferably the non-activated carbon composition portion is free of activated carbon, yet more preferably the non-activated carbon composition portion comprises titanium dioxide (preferably at the levels previously indicated), and yet even more preferably the non-activated carbon composition portion is free or substantially free of the interference pigment.

Many users who squeeze out of toothpaste tube that stripped with a black phase and a white phase find the aesthetics superior to at least a commercial available version that is notable without the subject interference pigments.

### Interference Pigments

The dentifrice compositions of the present invention comprise pigments exhibiting interference of colors dependent on angles of observation, so called color-shifting effects (“interference pigment”). Briefly, interference pigments manipulate incident light by means of refractions and reflection such that resultant refracted and reflected light generation color perception in the human eye. Base materials whose surface is coated with metal oxide having high and low refractive indexed which can be done once or many times. This coating process is by a wet chemical procedure that controls the optical thickness of the desired coating. An example of an interference pigment of the present invention is the XIRONA® product line of color travel pigments from Merck (Darmstadt, Germany) FA preferred product is XIRONA® Moonlight Sparks™ (product no. 17746) characterized as a silver-white powder with golden sparkle effect. For purposes of clarification, the RONASTAR® product line of pigments (also from Merck) is not within the scope of this invention. The interference pigment of the present invention have a particle size in microns from 1 to 500, preferably from 5 to 250, more preferably from 20 to 200, alternatively from 10 to 60, or 5 to 50. The interference pigment of the present invention has D-50 in microns as 10 to 100, preferably from 60 to 90. Examples of base material include mica or calcium aluminum borosilicate, preferably calcium aluminum borosilicate. Examples of metal oxides having a high refractive index are TiO₂, SnO₂, Fe₂O₃, Fe₃O₄, CoO, Co₂O₃, ZrO₂, Cr₂O₃, or their mixture or compounds, preferably TiO₂. Examples of metal oxides having a low refractive index are SiO₂, AlCl₃, AlCl₃, Al(OH)₃, B₂O₃, or their mixture or compounds, preferably SiO₂. Alternatively, the interference pigments are free of a base material (and comprise a metal oxide with a high refractive index and a metal oxide with a low refractive index). In one example, the interference pigment comprises from 50% to 75% (preferably 52-72%) of calcium aluminum borosilicate by weight of the interference pigment, 12% to 25% (preferably 15-23%) of silicon dioxide (SiO₂ or silica) by weight of the interference pigment, titanium dioxide (TiO₂) 10% to 25% (preferably 13-23%) of titanium dioxide by weight of the interference pigment, and optionally from 0% to less than 2% of tin oxide (SnO₂), alternatively from 0.01% to less than 2% of SnO₂, by weight of the interference pigment. In one example, the interference pigment color shifts between a gold color and a silver color.

The dentifrice composition comprises 0.01, 0.03, 0.05, 0.07, 0.09, 0.1, 0.11, 0.13, 0.15, 0.17, 0.2, or 0.3 of interference pigment by weight percentage of the least one activated carbon containing phase of the composition, preferably from 0.002% to 4%, more preferably from 0.01% to 1%, yet more preferably from 0.05% to 0.5%, of interference pigment by weight of the dentifrice composition.

Alternatively the dentifrice composition, as a whole, comprises from 0.001% to 2%, preferably from 0.01% to 1%, more preferably from 0.05% to 0.5%, of the interference pigment by weight of the dentifrice composition. Said dentifrice composition may or may not contain activated carbon. Said dentifrice composition may or may not contain more than one phase. Said dentifrice compositions may contain the interference pigments throughout the composition, i.e., in one or more phases, or those phases that contain activate carbon or do not.

### Humectants

The dentifrice composition may further comprise from 5% to 70%, preferably from 10% to 50%, more preferably from 20% to 40%, or combinations thereof; preferably at a humectant selected from the group consisting of sorbitol, glycerin, xylitol, butylenes glycol, propylene glycol, trimethyl glycine, and mixtures thereof; preferably glycerin and/or sorbitol; more preferably sorbitol. In one example, the humectant comprises from 25% to 40% by weight of the dentifrice composition of sorbitol.

### PEG

The dentifrice composition may comprise a polyethylene glycol (PEG), preferably comprising from 0.1% to 5% of the PEG by weight of the dentifrice composition, more preferably the PEG having an average molecular weight range of from 300 Da to 1,000 Da (or combination thereof). In an embodiment, the dentifrice composition comprises from 0.5% to 4%, or 0.75% to 3% of the PEG. The PEGs may have an average molecular weight range of from 300 Da to 1,000 Da, 400 Da to 800 Da, or 500 Da to 700 Da. PEG 12 is an example having an average molecular weight of 600 Daltons. PEGs are commercially available from such suppliers as Dow Chemical, Clariant, and BASF (New Jersey, USA).

### Thickening System

The dentifrice compositions of the present invention may comprise a thickening system. Preferably the dentifrice composition comprises from 0.5% to 4%, preferably from 0.8% to 3.5%, more preferably from 1% to 3%, yet still more preferably from 1.3% to 2.6%, by weight of the composition, of the thickening system. More preferably the thickening system comprises a thickening polymer, a thickening silica, or a combination thereof. Yet more preferably, when the thickening system comprises a thickening polymer, the thickening polymer is selected from a carboxymethyl cellulose, a linear sulfated polysaccharide, a natural gum, and combination thereof. Yet still more preferably, when the thickening system comprises a thickening polymer, the thickening polymer is selected from the group consisting of: (a) 0.01% to 3% of a carboxymethyl cellulose (“CMC”) by weight of the composition, preferably 0.1% to 2.5%, more preferably 0.2% to 1.5%, by weight of the composition, of CMC; (b) 0.01% to 2.5%, preferably 0.05% to 2%, more preferably 0.1% to 1.5%, by weight of the composition, of a linear sulfated polysaccharide, preferably wherein the linear sulfated polysaccharide is a carrageenan; (c) 0.01% to 7%, preferably 0.1% to 4%, more preferably from 0.1% to 2%, yet more preferably from 0.2% to 1.8%, by weight of the composition, of a natural gum; (d) combinations thereof. Preferably when the thickening system comprises a thickening silica, the thickening silica is from 0.01% to 10%, more preferably from 0.1% to 9%, yet more preferably 1% to 3%, by weight of the composition.

Preferably the linear sulfated polysaccharide is a carrageenan (also known as carrageenin). Examples of carrageenan include Kappa-carrageenan, Iota-carrageenan, Lambda-carrageenan, and combinations thereof.

In one example the thickening silica is obtained from sodium silicate solution by destabilizing with acid as to yield very fine particles. One commercially available example is ZEODENT® branded silicas from Huber Engineered Materials (e.g., ZEODENT® 153, 165, 165H, or combinations thereof). Abrasive silicas are discussed below.

In one example the CMC is prepared from cellulose by treatment with alkali and monochloro-acetic acid or its sodium salt. Different varieties are commercially characterized by viscosity. One commercially available example is Aqualon™ branded CMC from Ashland Special Ingredients (e.g., Aqualon™ 7H3SF; Aqualon™ 9M3SF Aqualon™ TM9A; Aqualon^{TM} TM12A).

Preferably a natural gum is selected from the group consisting of gum karaya, gum arabic (also known as acacia gum), gum tragacanth, xanthan gum, and combination thereof. More preferably the natural gum is xanthan gum. Xanthan gum is a polysaccharide secreted by the bacterium Xanthomonas camestris. Generally, xanthan gum is composed of a pentasaccharide repeat units, comprising glucose, mannose, and glucuronic acid in a molar ratio of 2:2:1, respectively. The chemical formula (of the monomer) is C₃₅H₄₉O₂₉. In one example, the xanthan gum is from CP Kelco Inc (Okmulgee, US).

Optionally one or more structuring agents may also be included, for example, selected from the group consisting of: a gum-type colloidal polymer; a cellulosic polymer; an acrylate polymer; and a clay or fine particulate.

### Viscosity

Preferably the dentifrice compositions of the present invention have a viscosity range from 150,000 centipoise to 850,000 centipoise (“cP”). A method for assessing viscosity is described. The viscometer is Brookfield® viscometer, Model DV-I Prime with a Brookfield “Helipath” stand. The viscometer is placed on the Helipath stand and leveled via spirit levels. The E spindle is attached, and the viscometer is set to 2.5 RPM. Detach the spindle, zero the viscometer and install the E spindle. Then, lower the spindle until the crosspiece is partially submerged in the paste before starting the measurement. Simultaneously turn on the power switch on the viscometer and the helipath to start rotation of the spindle downward. Set a timer for 48 seconds and turn the timer on at the same time as the motor and helipath. Take a reading after the 48 seconds. The reading is in cP.

pH

The pH of the dentifrice composition may be from 4 to 11, 5 to 10, or from 7 to 9. The pH is typically measured using a ratio of 1:3 of paste:water, whereby 1 gram of the dentifrice composition (e.g., toothpaste) is mixed into 3 grams of deionized water, and then the pH is assessed with a industry accepted pH probe that is calibrated under ambient conditions. The pH is measured by a pH meter with Automatic Temperature Compensating (ATC) probe. The pH meter is capable of reading to 0.001 pH unit.

After each usage the electrode should be washed free from the sample solution with water. Remove any excess water by wiping with a tissue. When electrode is not in use, keep electrode tip immersed in pH 7 buffer solution or electrode storage solution. Equipment details are as follows:

pH Meter: Meter capable of reading to 0.01 or 0.001 pH units.

Electrode: Orion Ross Sure-Flow combination: Glass body—VWR #34104-834/Orion #8172BN or VWR#10010-772/Orion #8172BNWP.
Epoxy body—VWR #34104-830/Orion #8165BN or VWR#10010-770/Orion #8165BNWP.
Semi-micro, epoxy body—VWR #34104-837/Orion #8175BN or VWR#10010-774/Orion #3175BNWP.
Orion PerpHect combination: VWR #34104-843/Orion #8203BN semi-micro, glass body.

ATC Probe: Fisher Scientific, Cat. # 13-620-16.

### pH Buffering Agent

The dentifrice compositions herein may include an effective amount of a buffering agent or pH trimming agents, as used herein, refer to agents that can be used to adjust the pH of the dentifrice compositions to the above-identified pH range. The buffering agents include alkali metal hydroxides, ammonium hydroxide, organic ammonium compounds, carbonates, sesquicarbonates, borates, silicates, phosphates, imidazole, and mixtures thereof.

Specific buffering agents include monosodium phosphate (monobasic sodium phosphate), trisodium phosphate (sodium phosphate tribasic dodecahydrate or TSP), sodium benzoate, benzoic acid, sodium hydroxide, potassium hydroxide, alkali metal carbonate salts, sodium carbonate, imidazole, pyrophosphate salts, sodium gluconate, lactic acid, sodium lactate, citric acid, sodium citrate, phosphoric acid.

In one embodiment, 0.01% to 3%, preferably from 0.1% to 1% of TSP by weight of the composition, and 0.001% to 2%, preferably from 0.01% to 0.3% of monosodium phosphate by weight of the composition is used. Without wishing to be bound by theory, TSP and monosodium phosphate may have calcium ion chelating activity and therefore provide some monofluorophosphate stabilization (in those formulations containing monoflurophospahte).

### Water

The term “orally acceptable carrier” as used herein means a liquid or semi-solid vehicle such as a paste or a gel for containing the active ingredients of the present invention and delivering them to the oral cavity. Water is commonly used as a carrier material in dentifrice compositions due to its many benefits. For example, water is useful as a processing aid, is benign to the oral cavity and assists in quick foaming of toothpastes. Water may be added as an ingredient in its own right or it may be present as a carrier in other common raw materials such as, sorbitol and sodium lauryl sulphate. The term total water content as used herein means the total amount of water present in the dentifrice composition, whether added separately or as a solvent or carrier for other raw materials but excluding that which may be present as water of crystallization in certain inorganic salts.

The dentifrice compositions of the present invention may comprise from 3% to 65%, preferablyl5% to 45%, or from 20% to 40%, by weight of the dentifrice composition, of a total water content. Preferably, the water is USP water.

### Chelant

The dentifrice compositions of the present invention may optionally comprise one or more chelants, also known as chelating agents. The term “chelant”, as used herein means a bi- or multidentate ligand having at least two groups capable of binding to metal ions and preferably other divalent or polyvalent metal ions and which, at least as part of a chelant mixture, is capable of solubilising the metal ions within the dentifrice composition. Groups capable of binding to metal ions include carboxyl, hydroxl and amine groups. Typically, those chelants used herein will also form water soluble stable complexes with metal ions.

### Anti-Microbial Agent

The dentifrice composition may comprise an anti-microbial agent, preferably from 0.01% to 5%, or from 0.1% to 1% of the anti-microbial agent by weight of the dentifrice composition. These anti-microbial agents may include a zinc ion source, paraben, Triclosan, phenyoxyethanol, mitormaldehyde donor, isopropyl methylphenol, or combination thereof.

### Fluoride Ion Source

The dentifrice composition may further comprise an effective amount of a fluoride ion source (e.g., for anti-carries benefit). In one example, the fluoride ion source may comprise one or a mixture of sodium fluoride, indium fluoride, amine fluoride or sodium monofluorophosphate (MFP). In another embodiment, the fluoride ion source is substantially free of a stannous fluoride. The fluoride ion source may be present in an amount sufficient to give a fluoride ion concentration in the composition at 25° C., and/or in one embodiment can be used at levels of from 0.0025% to 5% by weight of the dentifrice composition, alternatively from 0.005% to 2.0% by weight of the dentifrice composition, to provide anti-caries effectiveness.

In one embodiment, the fluoride ion source is sodium monofluorophosphate, and wherein the composition comprises 0.0025% to 2% of the sodium monofluorophosphate by weight of the composition. In another embodiment, the dentifrice composition comprises from 0.0025% to 2% of a fluoride ion source by weight of the composition.

### Abrasive

Dental abrasives are useful in dentifrice compositions for their ability to remove surface stains and pellicle and for polishing the teeth. The dentifrice compositions of the present invention may contain a dental abrasive. Dental abrasives used in the dentifrice composition of the subject invention include many different materials. The material selected must be one which is compatible with the composition of interest and does not excessively abrade dentin. Suitable abrasives include, for example, silicas including gels and precipitates, fused silica, insoluble sodium polymetaphosphate, hydrated alumina, and resinous abrasive materials such as particulate condensation products of urea and formaldehyde.

Silica dental abrasives of various types are preferred herein because of their unique benefits of exceptional dental cleaning and polishing performance without unduly abrading tooth enamel or dentine. Silica abrasive polishing materials herein, as well as other abrasives, generally have an average particle size ranging from 0.1 to 30 μm, and preferably from 5 to 15 μm. The abrasive can be precipitated silica or silica gels such as the silica xerogels marketed under the trade name “Syloid” by the W.R. Grace & Company, Davison Chemical Division and precipitated silica materials such as those marketed by the J.M. Huber Corporation under the trade name, Zeodent®, particularly the silicas carrying the designation Zeodent 103, 203, 108, 124, 120, 113, 115, 109, 119, and combination thereof.

Level of abrasive silica may include from 5% to 35%, or from 10 to 30%, or from 15% to 25%, by weight of the dentifrice composition. Preferably more than one type of abrasive silica is used.

The dentifrice composition of the present invention may comprise a total silica (abrasive silica, thickening silica, or combination thereof) from 5% to 45%, preferably from 10% to 40%, more preferably from 15% to 35%, yet more preferably from 17% to 30% of the silica by weight of the dentifrice composition

### Anti-Calculus Agent

The dentifrice compositions may include an effective amount of an anti-calculus agent, which in one embodiment may be present from 0.05% to 10%, 0.1% to 5%, or from 1% to 5%, by weight of the dentifrice composition. Non-limiting examples include those described in U.S. Publication No. 2011/0104081A1 at paragraph 64, and those described in U.S. Publication No. 2012/0014883A1 at paragraphs 63 to 68, as well as the references cited therein. One example is a pyrophosphate salt as a source of pyrophosphate ion. In one example, the composition comprises tetrasodium pyrophosphate (TSPP) or disodium pyrophosphate (also known as sodium acid pyrophosphate or disodium dihydrogen pyrophosphate or “SAPP”) or combinations thereof; preferably SAPP.

### Surfactant

The dentifrice compositions herein may include a surfactant. The surfactant may be selected from anionic, nonionic, amphoteric, zwitterionic, cationic, betaine surfactants, or mixtures thereof. The dentifrice composition may include a surfactant at a level of from 0.025% to 9%, 0.05% to 5%, 0.1% to 4%, 0.5% to 3%, or from 1% to 3% by weight of the total dentifrice composition. Non-limiting examples of anionic surfactants may include those described at US 2012/0082630 A1 at paragraphs 32, 33, 34, and 35. Non-limiting examples of zwitterionic or amphoteric surfactants may include those described at US 2012/0082630 A1 at paragraph 36; cationic surfactants may include those described at paragraphs 37 of the reference; and nonionic surfactants may include those described at paragraph 38 of the reference. Preferred surfactant is sodium lauryl sulfate (SLS).

### Flavorant

The dentifrice compositions herein may include from 0.01% to 5%, alternatively from 0.01% to 4%, alternatively from 0.1% to 3%, alternatively from 0.5% to 2%, alternatively combination thereof, of a flavor composition by weight of the dentifrice composition. Flavor ingredients may include those described in U.S. Patent Publication No. 2012/0082630A1. One example of a flavorant is a grapefruit flavor.

### Method of Use

The present invention also relates to methods for treating the oral cavity comprising administering to the oral care cavity a dentifrice composition according to the present invention.

In an embodiment, the term “treating” refers to cleaning and polishing teeth. The method of use herein comprises contacting a subject's dental enamel surfaces and oral mucosa with the dentifrice compositions according to the present invention. The method of treatment may be by brushing with a dentifrice or rinsing with as a dentifrice slurry. In a preferred embodiment, the dentifrice composition is a toothpaste.

### EXAMPLES

The following examples and descriptions further clarify embodiments within the scope of the present invention. These examples are given solely for the purpose of illustration and are not to be construed as limitations of the present invention as many variations thereof are possible without departing from the spirit and scope.

A composition according to the present invention is prepared.

TABLE 1
Formulation of two phase toothpaste of the present invention

| | Activated | Non-Activated |
|---|---|---|
| | Composition | Composition |
| | Portion | Portion |
| | (Phase 1) | (Phase 2) |
| Ingredient: | Weight % | Weight % |
| Coolant | 0.1 | 0.1 |
| Titanium Dioxide | 0 | 0.5 |
| Activated Carbon | 0.125 | 0 |
| Interference Pigment | 0.1 | 0 |
| (XIRONA ® Moonlight Sparks ™) | | |
| Flavor | 0.95 | 0.95 |
| Water | 31.5108 | 31.4283 |
| Sorbitol | 34.2594 | 34.0669 |
| Saccharin Sodium | 0.3 | 0.3 |
| NaOH | 0.85 | 0.85 |
| Carboxymethyl cellulose (CMC) | 0.8 | 0.8 |
| Sodium acid pyrophosphate (SAPP) | 3.32 | 3.32 |
| Silica | 23.5 | 23.5 |
| Sodium Lauryl Sulfate (SLS) | 2.4418 | 2.4418 |
| Polyethylene Glycol 600 | 1.5 | 1.5 |
| Sodium Fluoride | 0.243 | 0.243 |
| Total: | 100 | 100 |

Two user studies are conducted comparing the invention formula detailed in Table 1 with that of commercialized product brand DARLIE®, Super White Charcoal ( [symbol] ), Bamboo Flavor ( [symbol] ), Batch No. 20160511HH413A. This commercialized product has charcoal containing phase and a white (presumably titanium dioxide containing) This commercialized formulation is absent of Interference Pigments.

The first test is a visual having 49 users. The users are asked four questions to select which composition was preferred based on the question provided. The inventive composition out-performed the commercial product on all four attributes as detailed in Table 2.

TABLE 2
49 users are asked four questions as part of a visual test comparing the
inventive composition and a commercialized product.

| | Inventive | DARLIE ® | No |
|---|---|---|---|
| User Questions: | Composition | Toothpaste | difference |
| Which toothpaste is visually better? | 71% | 20% | 9% |
| | (35) | (10) | (4) |
| Which toothpaste appearance do | 69% | 22% | 9% |
| you like better? | (34) | (11) | (4) |
| Which toothpaste appearance is | 84% | 6% | 10% |
| more innovative and unique? | (41) | (3) | (5) |
| Which toothpaste looks more | 77% | 23% | 0% |
| attractive for you to brush with? | (38) | (11) | (0) |

The second test is a usage test having 60 users. The users are asked five questions to select which composition was preferred based on the question provided. The inventive composition out-performed the commercial product on all five attributes as detailed in Table 3.

TABLE 3
60 users are asked five questions as part of a usage test comparing the
inventive composition and a commercialized product.

| | Inventive | DARLIE ® | No |
|---|---|---|---|
| User Questions: | Composition | toothpaste | difference |
| Which toothpaste is better overall? | 60% | 35% | 5% |
| | (36) | (21) | (3) |
| Which toothpaste do you like | 62% | 32% | 6% |
| better? | (37) | (19) | (4) |
| Which toothpaste appearance is | 67% | 15% | 18% |
| more innovative and unique? | (40) | (9) | (11) |
| Which toothpaste is more premium? | 47% | 35% | 18% |
| | (28) | (21) | (11) |
| Which toothpaste is better in | 47% | 26.5% | 26.5% |
| appearance? | (28) | (16) | (16) |

The results indicate that users prefer the inventive composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A dentifrice composition comprising 0.001% to 2% of an interference pigment by weight of the composition.

2. The dentifrice composition of claim 1, further comprising at least one phase of an activated carbon composition portion, wherein the activated carbon composition portion comprises activated carbon.

3. The dentifrice composition of claim 2, wherein the activated carbon composition portion further comprises the interference pigment.

4. The dentifrice composition of claim 3, wherein the interference pigment comprises from 0.01% to 1% by weight of the activated carbon composition portion.

5. The dentifrice composition of claim 3, wherein the interference pigment color shifts between a gold color and a silver color.

6. The dentifrice composition of claim 3, wherein the interference pigment has a particle size from 5 to 250 microns.

7. The dentifrice composition of claim 3, wherein the interference pigment has a D-50 from 60 to 90 microns.

8. The dentifrice composition of claim 3, wherein the interference pigment comprises titanium dioxide.

9. The dentifrice composition of claim 8, wherein the interference pigment comprises from 10% to 25% titanium dioxide by weight of the interference pigment.

10. The dentifrice composition of claim 3, wherein the interference pigment comprises silicon dioxide.

11. The dentifrice composition of claim 10, wherein the interference pigment comprises from 12% to 25% silicon dioxide by weight of the interference pigment.

12. The dentifrice composition of claim 3, wherein the interference pigment comprises calcium aluminum borosilicate.

13. The dentifrice composition of claim 12, wherein the interference pigment comprises from 50% to 75% calcium aluminum borosilicate by weight of the interference pigment.

14. The dentifrice composition of claim 1, further comprising at least one non-activated carbon composition portion, wherein the non-activated carbon composition comprises titanium dioxide.

15. The dentifrice composition of claim 14, wherein the non-activated carbon composition portion comprises from 0.01% to 3% of the titanium dioxide by weight of the non-activated carbon composition portion.

16. The dentifrice composition of claim 1, further comprising at least a fluoride ion source.

17. The dentifrice composition of claim 1, further comprising a humectant, wherein the humectant is selected from the group consisting of glycerin, sorbitol, and combinations thereof.

18. The dentifrice composition of claim 1, having a viscosity from 150,000 centipoise to 850,000 centipoise.

19. The dentifrice composition of claim 1, further comprising a silica; wherein the total silica comprises from 5% to 45% by weight of the dentifrice composition.

20. The dentifrice composition of claim 1, further comprising a grapefruit flavor, wherein the grapefruit flavor comprises from 0.5% to 1.5% by weight of the dentifrice composition.
